(19) United States
(12) Patent Application Publication
(10) Pub. No.: US 2021/0307609 A1
(43) Pub. Date: Oct. 7, 2021

(54) IMPLANTABLE INTRAOCULAR PRESSURE SENSORS CONFIGURED AS CAPSULAR TENSION RINGS

(71) Applicant: Qura, Inc., Framingham, MA (US)

(72) Inventors: Douglas P. Adams, Sudbury, MA (US); Amitava Gupta, Roanoke, VA (US)

(21) Appl. No.: 17/262,504

(22) PCT Filed: Jul. 25, 2018
(86) PCT No.: PCT/US18/43753

§ 371 Date: Jan. 22, 2021

(51) Int. Cl. A61B 3/16; A61F 2/16

(52) U.S. Cl. CPC A61B 3/16; A61F 2240/001; A61F 2250/0001; A61F 2/1694

(57) ABSTRACT

Devices that include an intraocular pressure sensor and are also adapted to function as a capsular tension ring. The devices include an arcuate body, optionally with first and second free ends, the arcuate body comprising an arcuate antenna and an elastomeric coating layer disposed on the arcuate antenna, and an electronic module adapted to sense intraocular pressure.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Some challenges or difficulties with implanting an intraocular lens into a patient include compromised zonular integrity, compromised capsular bag or rupture of the posterior capsule. In 1991, Hara and co-workers were the first to publish the idea of inserting an endocapsular ring into the capsular bag when implanting an intraocular lens (“IOL”) to address one or more of these challenges. They used a closed ring made of soft silicone with a groove on its inner surface to receive the loops of an IOL. At about the same time, Nagamoto independently presented the concept of using an open ring made of rigid poly methyl methacrylate (“PMMA”) in order to maintain the circular contour of the capsular bag and thus avoid deformation or decentration of soft intraocular lenses. Implantation of a PMMA ring in human eyes was first reported in 1993. This ring was produced by Morcher and marketed under the name ‘capsular tension ring’ (“CTR”). It carried characteristic eyelets at its ends for atraumatic insertion and better manipulation (see FIG. 1). It was soon discovered that in addition to providing additional support to a capsule with compromised zonules (i.e., zonular dehiscence), CTRs also prevented migration of lens epithelial cells and thus retarded the onset of posterior capsular opacification (“PCO”). The various CTR designs differ significantly in resilience as defined by the spring constant, ranging from 0.88 to 4.55 mN/mm. While softer rings cause less zonular stress during insertion, more rigid rings counteract fibrotic capsular bag contraction. Some studies reported that, in cadaver eyes, a 12.5-mm ring diameter was found most appropriate for the human capsular bag. Commercially available CTRs range from 12.5 mm to 13.5 mm in diameter when designed for implantation in the capsule, while those designed for implantation in the ciliary sulcus have larger diameters, ranging from 14.0 mm to 14.5 mm. They are selected so that they fit the capsular equator of the individual patient, or to provide a desired level of centrifugal force, depending on their expected function in a particular case. They are typically, though not always, ring shaped and the thickness of the ring ranges from 0.12 mm to 0.7 mm when made of PMMA.

FIGS. 1A-1I show photomicrographs of exemplary capsular tension rings marketed by Morcher. The standard Morcher CTR comes in three sizes based on uncompressed diameter: 12.3 mm (compresses to 10 mm, Morcher 14, used for axial length <24 mm); 13 mm (compresses to 11 mm, Morcher 14 C, used for axial length of 24-28 mm); and 14.5 mm (compresses to 12 mm, Morcher 14A, used for axial length >28 mm, designed for implantation in the sulcus). The Henderson CTR7 (FC-CBR) from Morcher GmbH (shown in FIG. 1I), differs from the standard ring in that it has eight equally spaced indentations of 0.15 mm and an uncompressed diameter of 12.29 mm that is compressible to 11 mm. It has been reported that an advantage of the Henderson CTR is that it allows for easier removal of nuclear and cortical material while maintaining equal expansion of the capsular bag.

A number of clinical studies of the safety and efficacy of CTR in the human eye have been performed. In all cases, no evidence of lack of biocompatibility have been presented, including breach of the capsule, or excessive IOL decentration, dislocation or rotational displacement of the IOL.

There is a benefit for some patients to have an implanted sensor in one or both eyes, such an intraocular pressure sensor. This can be the case with subjects with, for example, pre-existing glaucoma, preexisting diabetes (DM), or other retinal diseases such as diabetic retinopathy. A recent meta-analysis of 47 studies by Zhao and colleagues reported a pooled relative risk of glaucoma of 1.48 in patients with diabetes compared to those without diabetes. In addition, there was an increasing relative risk of glaucoma that was positively associated with diabetes duration. Though elevated IOP alone is a significant risk factor for but is not diagnostic for glaucoma, diabetic patients had a pooled average increase in IOP of 0.09 mmHg for every 10 mg/dl increase in fasting glucose. An epidemiological study on Danish patients suffering from diabetes indicated a strong association between occurrence of Diabetes Mellitus and onset of glaucoma treatment among the entire Danish population.

Therefore, a need exists for devices that are sized and configured to be implanted in an eye, adapted to sense pressure, and are also sized and configured to function as a capsular tension ring.

### SUMMARY OF THE DISCLOSURE

The disclosure relates to implantable intraocular pressure (“IOP”) sensing devices that are configured, sized and adapted such that they also function as capsular tension rings. The sensing devices can be implanted into the capsule or outside of the capsule (e.g., in the ciliary sulcus) during a cataract extraction and intraocular lens implantation procedure. The devices integrate intraocular pressure sensing with capsular tension ring function. The devices include an arcuate body (e.g., an annular, or partially annular body), and a pressure sensing unit or subassembly, at least a portion of which can extend outside of the annular or partially annular body configuration. In some embodiments the pressure sensing unit is positioned completely within the annular or partially annular body. A pressure sensing unit can be referred to herein as an electronic module.

Preferably, the IOP sensing devices can have physical properties that are similar to existing capsular tension rings, so that when the devices are implanted they can function like known capsular tension rings. The arcuate body (e.g., annular, or partially annular body) portions of the devices are thus designed carefully to control their physical properties. For example, the IOP sensing devices should have stiffnesses that are at least similar to existing capsular tension rings, so that when implanted they can function to, if implanted inside a capsular bag, apply sufficient radially outward forces against the equatorial region of the bag. Additionally, the stiffness of the capsular tension rings integrated with a sensor is further adjusted to bear the weight of the coupled sensor body that comprise an electronic module and an antenna body without deforming or buckling inside the capsule of the eye.

### BRIEF DESCRIPTION OF THE DRAWINGS

FIGS. 1A-1I illustrate exemplary prior art capsular tension rings.

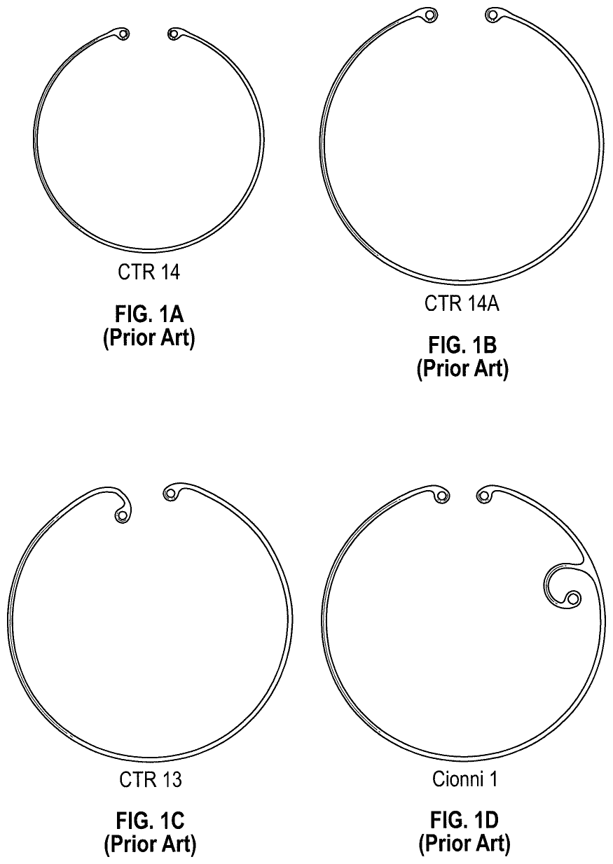

FIG. 2 illustrates an exemplary implantable pressure sensing device that is adapted to also function as a capsular tension ring.

FIG. 3 illustrates an exemplary implantable pressure sensing device that is adapted to also function as a capsular tension ring.

FIG. 4 is an exemplary method of manufacturing an implantable pressure sensing device that is also adapted to function as a capsular tension ring.

### DETAILED DESCRIPTION

The disclosure is related to implantable intraocular pressure (“IOP”) sensing devices that are configured, sized and adapted such that they also function as capsular tension rings. The devices are described herein as being configured, sized and adapted to be implanted in a capsular bag or ciliary sulcus, and can be implanted during a cataract extraction and intraocular lens implantation procedure. The devices can thus function as both intraocular pressure sensors and capsular tension rings.

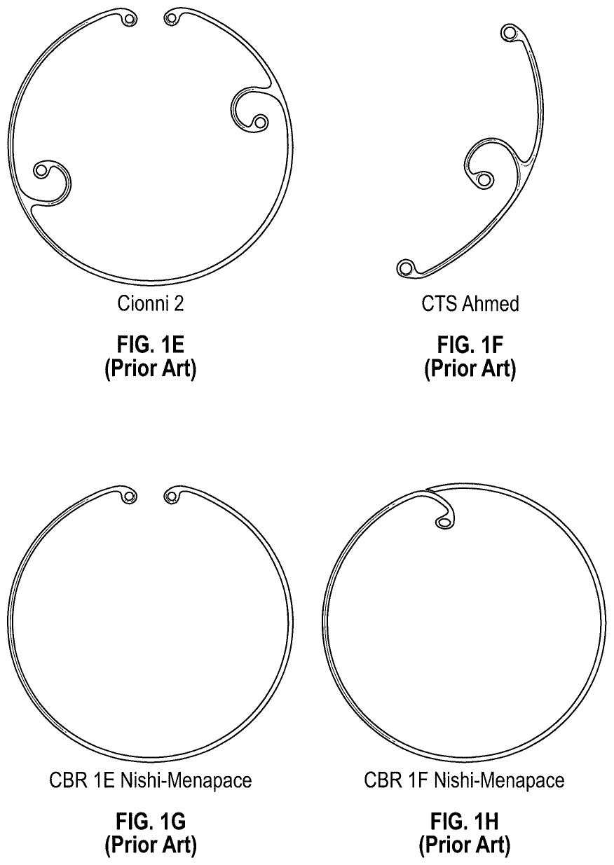

FIG. 2 illustrates an exemplary side view of an exemplary implantable IOP sensing device that is also configured and adapted to function as a capsular tension ring. Device 10 includes an arcuate body 12 (which in this embodiment has a partially annular configuration) and a pressure sensing unit 14. The partially annular body 12 includes first end 11 and second end 13, which together form two free ends. In alternative designs, the partially annular body could be replaced with an annular body, which would not have free ends. Partially annular body 12 includes antenna 15, which has a partial loop, or ring, configuration, and a coating layer 16 that is disposed on antenna 15. Antenna 15 is in operable communication with pressure sensing unit 14, which provides functionality described below. In FIG. 2, pressure sensing unit 14 and partially annular body 12 are integrated into a single hermetically sealed system.

When the phrase “arcuate body” is used herein, it is intended that this phrase includes at least partially annular bodies and annular bodies.

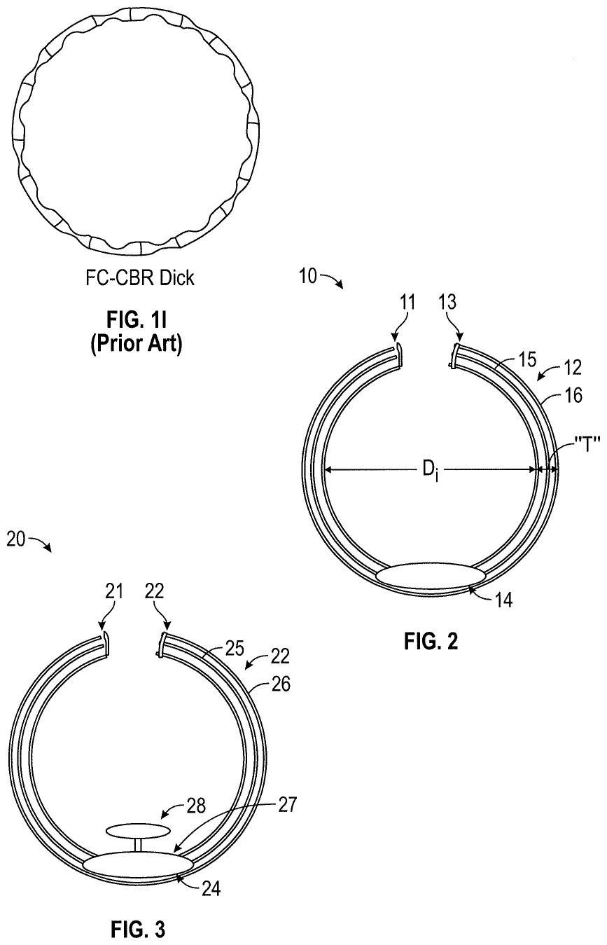

FIG. 3 illustrates an exemplary implantable IOP sensing device that is also configured and adapted to function as a capsular tension ring. Device 20 includes an arcuate body 22 (which in this embodiment has a partially annular configuration) and a pressure sensing unit 24. The partially annular body 22 includes first end 21 and second end 23, which together form two free ends. In alternative designs, the partially annular body could be replaced with an annular body, which would not have free ends. Partially annular body 22 includes antenna 25, which has a partial loop, or ring configuration, and a coating layer 26 that is disposed on antenna 25. Antenna 25 is in operable communication with pressure sensing unit 24, which provides functionality described below. In device 20, pressure sensing unit 24 includes first hermetically sealed member 27 and second hermetically sealed member 28. Second hermetically sealed member 28 is disposed outside of the coating layer on the annular body.

As set forth above, the arcuate body preferably comprises materials and thicknesses that provide physical properties that resemble existing capsular tension rings. This means that the material and dimensions of the antenna and coating layers are chosen that will provide the desired physical properties for the annular body.

The coating layers herein may be made of a silicone or acrylic elastomer, for example, silastic rubber or a copolymer of acrylates and methacrylates, cross-linked in order to preserve a an arcuate shape. In some preferred embodiments, the coating layers here are made of a cross-linked copolymer of acrylates and methacrylates. Its glass transition temperature can preferably be less than 10 C, and can be in the range 0 C to 10 C, so that the coating layer or layers are elastomeric during use at normal eye temperature (35-38 C).

The enclosed antennas herein act as a stiffener for the arcuate body, therefore use of PMMA for the coating layer should be generally avoided when constructing the arcuate bodies of the devices here. The thickness of the applied coating and the material composition (including, without limitation, its glass transition temperature and its tensile and bulk moduli) are adjusted so that the spring constant of the resulting CTR does not exceed 4 mN/mm.

Preferably, the antenna with its substrate make a snug fit with the surrounding elastomeric layer, which together make up the structure that functions as the CTR, so that there is no free space between the antenna and the coating layer that may otherwise accumulate moisture or aqueous humor.

In some preferred embodiments, the modulus of the arcuate body portion of the device should be in the range 1-10 MPA, and its elongation at break should be in the range 50-150%. The spring constant of the arcuate body portion, including the enclosed antenna, should be in the range 2.00-4.00 mN/mm.

In some embodiments, the arcuate body is formed by first making (e.g., casting) a hollow tubular element out of, for example, an acrylic or silicone elastomer, then advancing the antenna inside the formed hollow tubular element. The tubular element and antenna are sized such that the antenna makes a snug fit with the inner surfaces of the hollow tubular element. The device can have an internal diameter “D” (see FIG. 2) in the range 10.0-15.0 mm, more preferably 11.0 mm to 14.0 mm. The wall thickness of the at least partially annular body should be in the range of 0.1 mm to 0.25 mm. The internal diameter of a hollow tubular element (if that is incorporated in the at least partially annular body) should be in the range of 0.1 mm to 0.20 mm.

In some preferred embodiments, the pressure sensing units herein can be hermetically sealed in a Titanium casing of thickness not exceeding 50 microns, and preferably in the range of 10-15 microns. The pressure sensing units can be encased in a multilayer coating comprised of SiOₓ and Paralyne (preferably Paralyne C), and is immersed in a substantially low viscosity liquid medium inside the hermetic seal. Preferably, the number of layers of coating applied is more than five, and the coating is in the range of 5-100 microns. Preferably, each such layer has a thickness of 5-100 nanometers. Preferably, the viscosity of the medium in which the sensor is immersed should not exceed 1000 cst at room temperature, and more preferably in the range 50-500 cst at 25 C. The hermetically sealed electronics package and the pressure sensor of the pressure sensing unit are preferably overcoated with a thin layer of the same copolymer material that is used to coat the antenna, so that there is no weld or adhesive joint between the at least partially annular body and the pressure sensing unit.

In some preferred embodiments, the entire device, including the at least partially annular body and the electronics, is coated with a highly biocompatible coating that prevents cellular deposition and minimizes fibrosis. Preferably, this coating is a hydrophilic cross-linked acrylate and/or methacrylate, made of polyethylene glycol segments.

Any of the pressure sensing units herein can include an intraocular pressure sensor, which can be, for example, piezoresistive or capacitative. The pressure sensing units can also include a microcontroller or an ASIC with embedded firmware to provide electrical control functions. The pressure sensing units can also include a real time clock, a voltage converter, a rechargeable battery, optionally a thin film solid state rechargeable battery. A rechargeable battery may either be integrated into one hermetically sealed package or multiple sealed packages connected by electrical wires conveyed into each such package by vias (see, for example, FIG. 3). The pressure sensing units can also include a flash memory and an EEPROM. The pressure sensing units can be thought of as being in operable communication with the antennas herein, even though the antennas herein can be thought of a part of the overall pressure sensing devices. The pressure sensing units can also include one or more elements adapted to wirelessly transfer data and power to and from the implantable device to an external device. The electronics module comprising the pressure sensing unit may be controlled and operated by firmware that includes embedded algorithms stored in the EEPROM memory unit of the pressure sensor. Preferably the firmware is reprogrammable via wireless means remotely by an external unit.

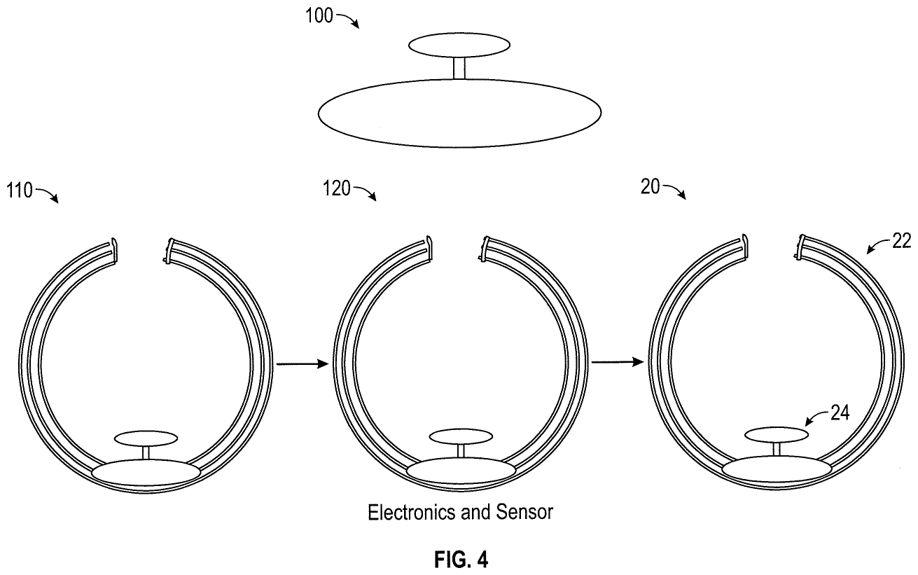

Exemplary Manufacturing Processes. There are several methods that can be used to fabricate the devices herein, including, for example, 3D printing, cast molding, injection molding, machining, etc. A merely exemplary process to make the device from FIG. 3 is illustrated in FIG. 4. In this approach, the pressure sensing unit and antenna 100, fabricated initially with straight antennas, is coated with a polymer that includes acrylates and methacrylates. This coating step can be accomplished by dipping or spraying the device with a mix of monofunctional acrylates and methacrylates and an initiator, so that an elastomeric coating is formed that remains uncross-linked. An uncross-linked coating has no shape memory, so it can be bent and shaped into the configuration as shown as device 110. A mix of a difunctional, trifunctional or tetrafunctional monomers and a UV photoinitiator is then sprayed on top of the uncross-linked coating, and the mixture is allowed to diffuse into the bulk of the coating. Device 110 is then exposed to actinic UV radiation, exposure to which activates the initiator, and initiates cross-linking. When the cross-linking process is completed, the shape of the at least partially annular body is now set, as shown as device 120. The cross-linking process can thus be used to set the configuration of the at least partially annular body. A biocompatible coating is then applied to device 120, resulting in device 20.

The two-step polymerization and forming process illustrated in FIG. 4 ensures that there is no free space between the inner wall of the coating layer and the antenna, which ensures there isn't any space for moisture or aqueous humor to accumulate.

The shaped antenna and coating thus provide capsular tension ring functionality to the implantable device, and the electronics sensing unit 24 includes the sensor module. A monomer application process, including without limitation, spraying, dipping or 3D printing is selected that provides the required level of uniformity in thickness of the coating prior to being polymerized in place. Variation in thickness (outer diameter) of the coating layer of up to +/−30% is generally acceptable, preferably being 200 microns +/−30%.

Any of the antennas herein can be made of gold, gold coated Nitinol, or gold-coated copper, and can have a thickness in the range of 25-100 microns. The antennas can have a circular cross section.

The thickness of the polymeric coating layer can be in the range of 40-235 microns, preferably in the range of 75-200 microns.

Table 1 provides examples of monomer compositions used in the exemplary process described with reference to FIG. 4.

| Exemplary | |
|---|---|
| Manufacturing steps | Exemplary monomer compositions |
| Uncross-linked | Isobutyl acrylate, ethyl acrylate, phenyl |
| coating formation | acrylate, phenoxyethyl acrylate, isobornyl |
| | acrylate, ethyl methacrylate, photoinitiators |
| | that initiate free radical polymerization |
| | including benzophenone derivatives, |
| | acetophenone derivatives, phosphine oxide |
| | derivatives, including TPO, TPO-L |
| Cross-linking coating | Ethylene glycol dimethacrylate, bisphenol A |
| to form at least | Diacrylate, trimethylene propane triacrylate, |
| partially annular body | pentaerythritol tetracrylate, TPO, TPO-L |
| Biocompatible coating | Ethylene glycol Diacrylate, ethylene glycol |
| formation | dimethacrylate, TPO, TPO-L |

The implantable pressure sensing devices can be adapted to communicate with an external unit capable of communicating with the implant wirelessly. The external unit can be adapted so that the external device can provide wireless energy transfer from and to the implant, can be capable of downloading sensed TOP data from the implant, can be adapted to perform data processing, can store data on board, and can be adapted to transmit the data to a database, optionally established in a cloud based server (ETD).

Any and all aspects of the implantable pressure sensing devices and methods of manufacture described in WO2017/210316 are fully incorporated by reference herein, and can be incorporated into any of the suitable implantable pressure sensors and method of manufacture herein.

1. An intraocular pressure sensor that is adapted to function as a capsular tension ring, comprising:
an arcuate body with first and second free ends, the arcuate body comprising an arcuate antenna and an elastomeric coating layer disposed on the arcuate antenna; and
an electronic module adapted to sense intraocular pressure.

2. The device of claim 1, wherein the arcuate body has an at least partially annular configuration, and the arcuate antenna has an at least partially annular configuration.

3. The device of claim 1, wherein the electronic module is in communication with the arcuate antenna.

4. The device of claim 3, wherein the coating layer is disposed over electronic module and the antenna.

5. The device of claim 1, wherein the coating layer comprises a cross-linked polymer, optionally with a glass transition temperature in the range 0 C to 10 C.

6. The device of claim 1, wherein the thickness of the coating layer is 50 microns to 400 microns, optionally 50 microns to 150 microns.

7. The device of claim 1, wherein the thickness of the antenna is 25 microns to 200 microns.

8. The device of claim 1, wherein the antenna is made from gold or gold-coated titanium.

9. The device of claim 1, wherein the coating layer comprises at least one of a silicone or an acrylic elastomer.

10. The device of claim 9, wherein the coating layer comprises a copolymer of at least one acrylate and at least one methacrylate, optionally with a glass transition temperature less than 10 C.

11. The device of claim 1, wherein the arcuate body has a diameter from 9.0 mm to 16.0 mm, optionally from 11.0 mm to 14.0 mm.

12. The device of claim 1, wherein a thickness (“T”) of the arcuate body is from 50 microns to 300 microns, optionally 50 microns to 100 microns.

13. The device of claim 1, wherein an internal diameter of the coating layer is in the range of 0.1 mm to 0.20 mm.

14. The device of claim 1, further comprising an outermost biocompatible coating.

15. The device of claim 1, wherein the coating layer is chemically bonded to the surface of the arcuate antenna.

16. The device of claim 1, wherein the arcuate body is adapted to be compressed through an incision made during a procedure that implants an intraocular lens.

17. An implantable pressure sensing device, comprising:
an arcuate body comprising an arcuate antenna and a coating layer disposed on the arcuate antenna, wherein a thickness of the coating layer on the arcuate antenna is from 50 microns to 400 microns;
an electronic module adapted to sense intraocular pressure.

18. The device of claim 17, wherein the coating is an elastomeric coating.

19. The device of claim 17, wherein the arcuate body has an at least partially annular configuration, and the arcuate antenna has an at least partially annular configuration.

20. The device of claim 17, wherein the electronic module is in communication with the arcuate antenna.

21. The device of claim 20, wherein the coating layer is disposed over the electronic module and the arcuate antenna.

22. The device of claim 17, wherein the thickness of the coating layer on the arcuate antenna is from 50 microns to 150 microns.

23. The device of claim 17, wherein the coating layer comprises a cross-linked polymer, optionally with a glass transition temperature in the range 0 C to 10 C.

24. The device of claim 17, wherein the thickness of the antenna is 25 microns to 200 microns.

25. The device of claim 17, wherein the antenna is made from gold or gold-coated titanium.

26. The device of claim 17, wherein the coating layer comprises at least one of a silicone or an acrylic elastomer.

27. The device of claim 26, wherein the coating layer comprises a copolymer of at least one acrylate and at least one methacrylate, optionally with a glass transition temperature less than 10 C.

28. The device of claim 17, wherein the arcuate body comprises an at least partially annular body that has a diameter from 9.0 mm to 16.0 mm, optionally from 11.0 mm to 14.0 mm.

29. The device of claim 17, wherein a thickness of the arcuate body is from 50 microns to 300 microns, optionally 50 microns to 100 microns.

30. The device of claim 17, wherein an internal diameter of the coating layer is in the range of 0.1 mm to 0.20 mm.

31. The device of claim 17, further comprising an outermost biocompatible coating.

32. The device of claim 17, wherein the coating layer is chemically bonded to the surface of the arcuate antenna.

33. The device of claim 17, wherein the arcuate body is adapted to be compressed through an incision made during a procedure that implants an intraocular lens.

34. A method of manufacturing an implantable intraocular pressure sensing device, comprising:
providing a straight antenna coupled to a pressure sensing unit;
applying at least one monomer, an initiator, and a cross linker to an outer surface of the straight antenna and an outer surface of the pressure sensing unit so that the at least one monomer is not cross linked;
deforming the straight antenna into an arcuate configuration;
exposing the monomer, initiator and cross-linker to cross-linking radiation to cross-link the at least one monomer, and thereby maintain the deformed arcuate configuration of the antenna.

35. The method of claim 34, wherein applying at least one monomer comprises applying at least one acrylate and at least one methacrylate.

36. The method of claim 34, further comprising applying a photoinitiator onto the uncross-linked coating after the deformation step and before the exposing step.

37. The method of claim 34, further comprising applying a biocompatible coating onto the maintained and deformed arcuate configuration.
